(19) United States
(12) Patent Application Publication
(10) Pub. No.: US 2008/0269647 A1
(43) Pub. Date: Oct. 30, 2008

(54) Device for Directing Ultrasound at a Target Region in a Human or Animal Body

(76) Inventor: Hugo Willem Brunsveld Van Hulten, Voorburg (NL)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/630,709

(22) PCT Filed: Jun. 22, 2005
(86) PCT No.: PCT/NL2005/000450

§ 371 Date: Mar. 28, 2008

(30) Foreign Application Priority Data
Jun. 24, 2004 (NL) 1026492

(51) Int. Cl. A61N 7/00

(52) U.S. Cl. 601/2

(57) ABSTRACT

The invention relates to a device for directing ultrasound at a target region in a human or animal body, in particular for treating proliferative tissue, comprising imaging means for generating one or more images of the target region in the body that is to be treated; processing means for processing the obtained images; at least one ultrasound transducer arranged for placement in direct proximity of the target region to be treated; and driving means for driving said at least one ultrasound transducer on the basis of signals generated by the processing means. The object of the invention is to provide a device as referred to in the introduction that enables a more precise orientation of the ultrasound transducer relative to the target region. According to the invention, the device is to that end provided with positioning means for orienting the ultrasound transducer in the three-dimensional space relative to the target region.

The invention relates to a device for directing ultrasound at a target region in a human or animal body, in particular for treating proliferative tissue, comprising imaging means for generating one or more images of the target region in the body that is to be treated; processing means for processing the obtained images; at least one ultrasound transducer arranged for placement in direct proximity of the target region to be treated; and driving means for driving said at least one ultrasound transducer on the basis of signals generated by the processing means.

Such a device is known, for example from International patent application WO 03/097162. According to said patent publication, the ultrasound transducer is driven to direct ultrasound at a specific target region within the framework of a treatment scheme. Said directing of ultrasound at the target region, however, only takes place by driving the transducer elements of the ultrasound transducer, which has a limiting effect as regards an optimum treatment on the basis of treatment schemes.

The object of the invention is to provide a device as referred to in the introduction that enables a more precise orientation of the ultrasound transducer relative to the target region. According to the invention, the device is to that end provided with positioning means for orienting the ultrasound transducer in the three-dimensional space relative to the target region.

This makes it possible to carry out more precise treatments with ultrasound of the target region by means of the device according to the invention; in particular the more precise positioning makes it possible to prevent ultrasound being applied to healthy tissue surrounding the target region (proliferative tissue), which is undesirable in particular when sensitive organs are concerned.

In one embodiment according to the invention, the positioning means comprise at least one excitable piezoelectric motor. Motors of this type are characterized by a very high degree of control and positioning precision.

More specifically, means for feeding back the current position of the motor to the driving means are provided near each piezoelectric motor, which feedback means in particular comprise at least one strain gauge. Said feedback makes it possible to realise a more precise device according to the invention, which is capable also of carrying out complex treatment schemes with ultrasound in a precise manner without any adverse consequences for the surrounding tissue.

In a special embodiment, the device is furthermore provided with Peltier-type cooling means.

Furthermore, the device may comprise a housing, in which at least the ultrasound transducer and the driving means are accommodated. As a result, a very compact construction is realised, which can be used for special ultrasound treatments.

More specifically, the housing may be mounted in a positioning or fixing frame for positioning or fixing at least one breast of a female patient, which functions as the target region. The ultrasound transducer can be mounted in the housing in such a manner that the transducer will direct ultrasound in lateral direction at the target region during operation.

According to a further aspect of the invention, the ultrasound transducer is a phased array transducer, more specifically, the ultrasound transducer is a multichannel transducer, in particular a 256-channel transducer. The use of a sufficiently large number of transducer elements makes it possible to direct and move the focus of the multichannel transducer at/over the target region in the human or animal body.

In another functional embodiment, the device according to the invention comprises a dose-planning unit, wherein the positioning means can be driven by the driving means partially on the basis of the signals as generated and delivered to the driving means by the dose-planning unit.

The device according to the invention makes it possible to carry out specific treatment methods in that the driving means are arranged for focussing the ultrasound transducer in predetermined planes relative to the target region. On the other hand, the driving means are arranged for focussing the ultrasound transducer spirally relative to the target region so as to make it possible to carry out another specific treatment method. The driving means may furthermore be arranged for volume-wise focussing of the ultrasound transducer relative to the target region.

Furthermore, the imaging means may be magnetic resonance-type imaging means.

The invention will now be explained in more detail with reference to the drawings, in which:

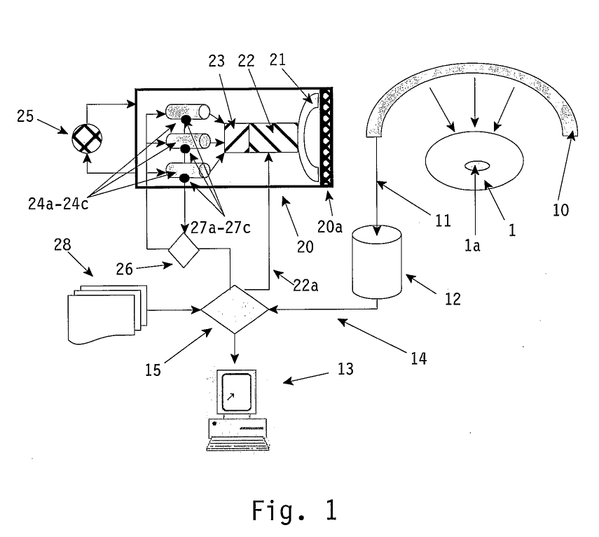

FIG. 1 is a schematic representation of a first embodiment of a device according to the invention;

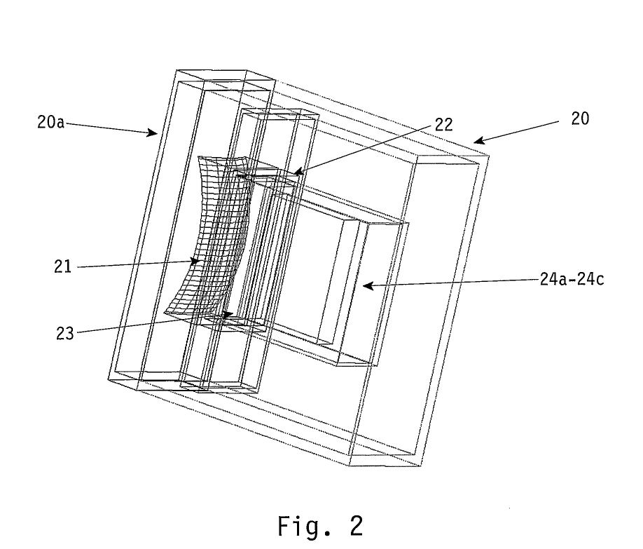

FIG. 2 is a partial view of the embodiment that is shown in FIG. 1;

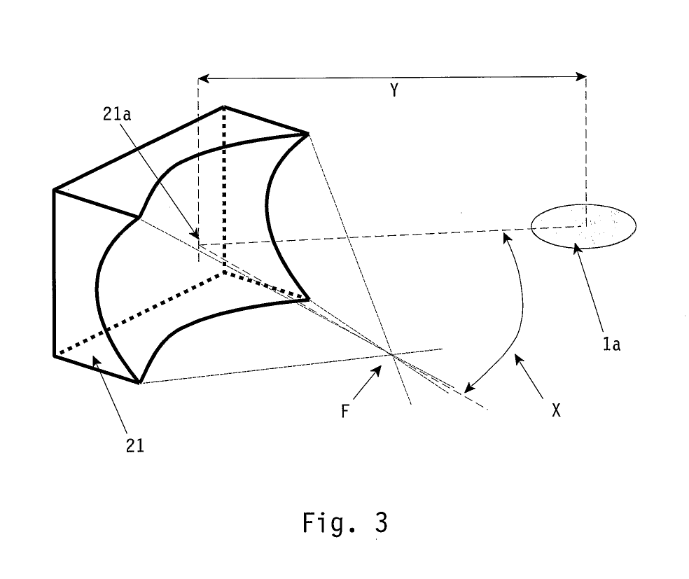

FIG. 3 is a schematic representation of the device according to the invention disposed in a treatment space;

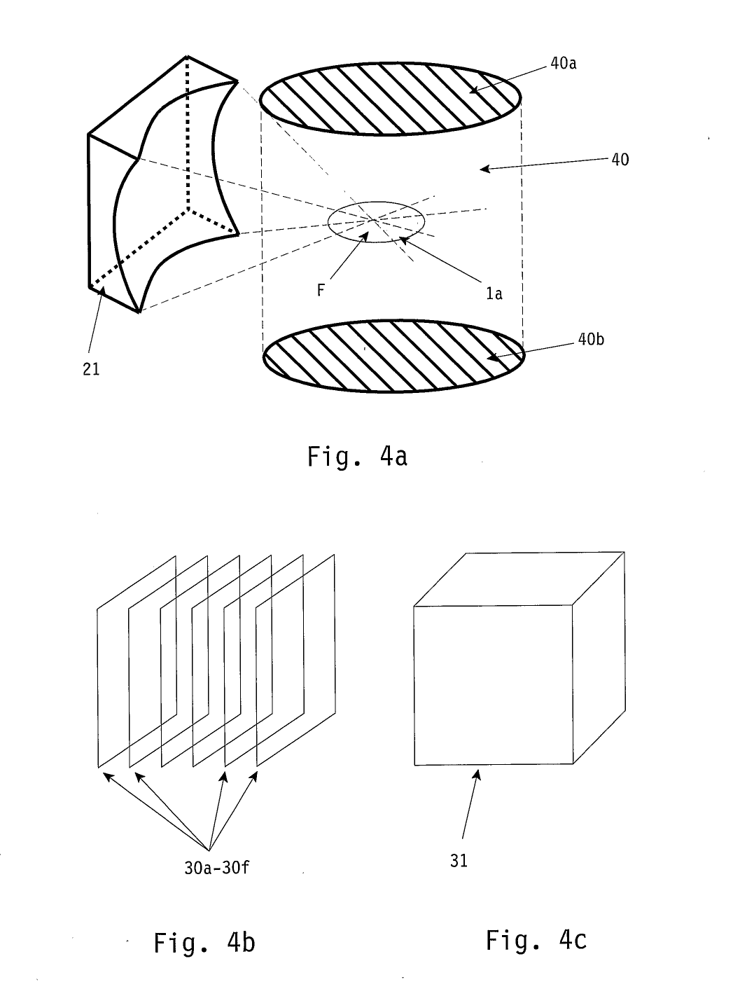

FIGS. 4a-4c show other applications of the device according to the invention in the treatment space;

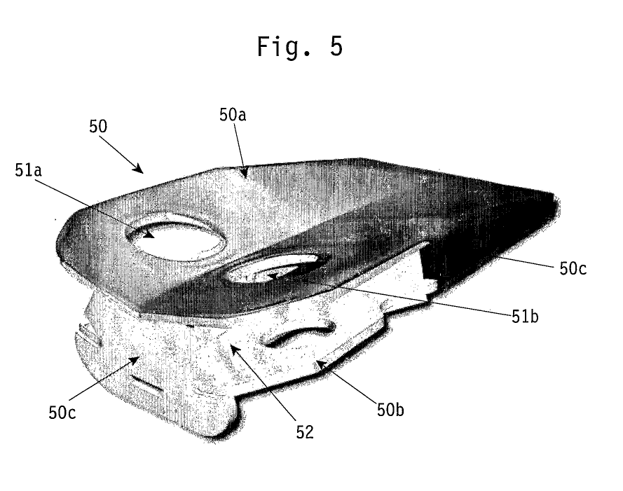

FIG. 5 shows auxiliary means to be used with the device according to the invention;

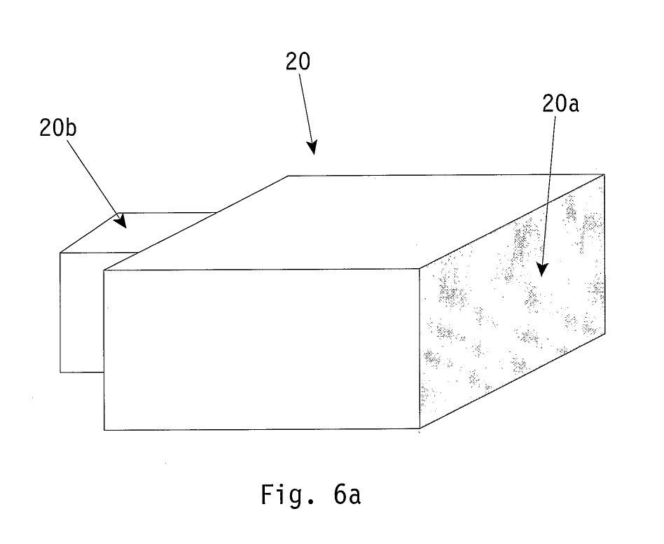

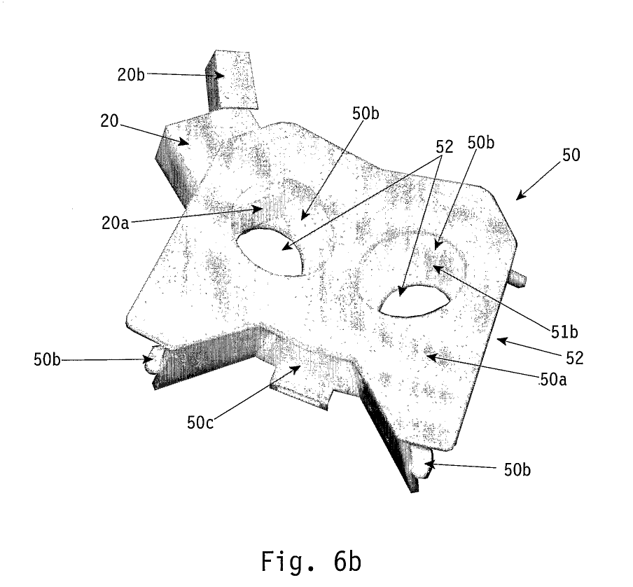

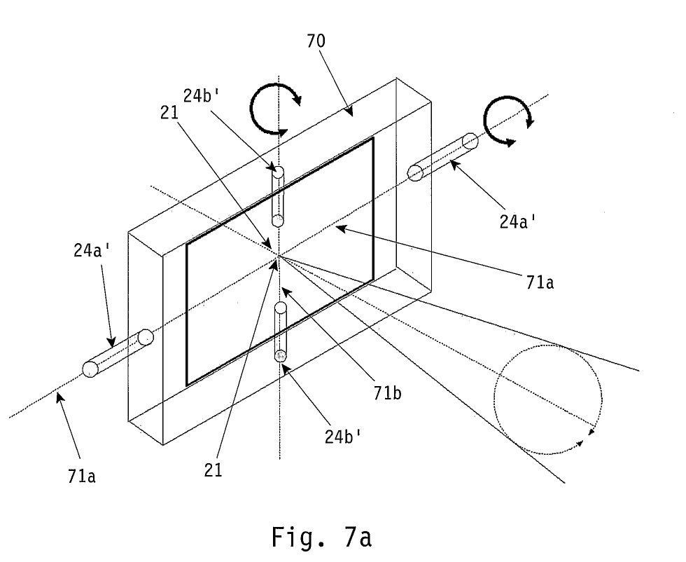

FIGS. 6a-6b show another embodiment of the device according to the invention with the auxiliary means of FIG. 5;

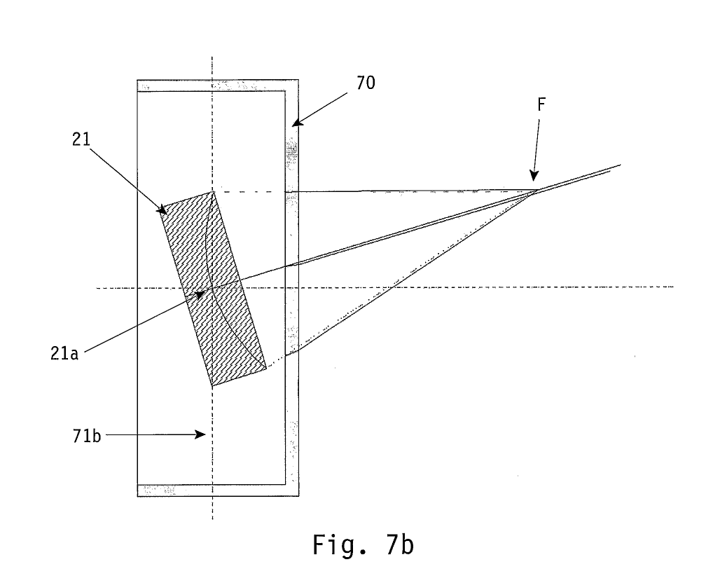

FIGS. 7a-7b show another embodiment of the device according to the invention.

Like parts will be indicated by the same numerals in the description below.

In FIG. 1 there is shown an embodiment of the device for treating a target region 1a in the human or animal body 1 by means of ultrasound.

The device 20 according to the invention is based on the treatment of the target region 1a with ultrasound that is generated in an ultrasound transducer 21, which ultrasound can escape from the device 20 via an application window 20a.

Preferably, the ultrasound transducer 21 is a multichannel transducer, in particular a 256-channel transducer. Said 256-channel ultrasound transducer is preferably of the phased array-type comprising 256 transducer elements, which are each individually driven by means of 256 signal channels. Said 256 signal channels are schematically indicated at 22. The large number of transducer elements makes it possible to orient and move the focus F of the multichannel transducer 21 precisely over the target region 1a in the human or animal body 1 for the purpose of carrying out the treatment scheme.

The 256 signal channels 22 are driven by a driving unit 15 via a suitable signal link 22a. The driving unit 15 is in turn driven by signal data obtained by imaging means 10, which produce one or more images of the body 1 (the patient), more in particular the target region 1a. In this specific embodiment, said imaging means 10 are MRI means, which generate digital images of the target region 1a on the basis of magnetic resonance. Said digital signal data are passed on to a data processing unit 12 via a suitable signal link, e.g. a local network, in which data processing unit the image data are converted into suitable drive signals that are carried to the driving unit 15 via the link 14a.

The device furthermore comprises a dose-planning unit 28, which generates a suitable dose planning on the basis of predetermined data, such as the image data generated by the imaging means 10, dose distributions of the ultrasound transducer 21 and additional arithmetic models, such as models that describe the propagation of waves through the body 1, as well as models that describe the generation and transportation of heat through the tissue 1.

Based on the image data as realised by the imaging means 10 and the dose planning as generated by the dose planning unit 28, the driving unit 15 drives the various transducer elements of the ultrasound transducer 21a via the signal link 22a. The driving unit 15 furthermore drives positioning means 26 for orienting the transducer element 21 precisely relative to the target region 1a. According to the invention, the positioning means 26 to that end comprise one or more excitable piezoelectric motors 24a-24c, which, depending on the manner in which they are driven, impose a movement on the ultrasound transducer 21 that forms part of the device 20. To that end, the ultrasound transducer 21 is according to the invention mounted in a housing 23, on which the various piezoelectric motors 24a-24c impose a random desired orientation in the three-dimensional plane relative to the target region 1a.

To feed the current position of the various motors 24a-24c (and consequently the ultrasound transducer 21) back to the driving unit 15, and more in particular to the positioning means 26, each motor 24a-24c is provided with a sensor 27a-27c, which registers the current position of each motor 24a-24c and feeds its back to the positioning means 26. In this specific embodiment, said sensors 27a-27c may be made up of at least one strain gauge.

The device 20 is furthermore provided with cooling means 25, more in particular of the Peltier type. Thus, the heat that is generated while the ultrasound transducer 21 is being driven can easily be dissipated, so that it will not affect the treatment scheme of the target region 1a.

Furthermore, the entire device may be adjusted and controlled via a control unit 13, for example a personal computer.

FIG. 2 shows the compact configuration of the device according to the invention, which device 20 is of compact construction, i.e. comprising one housing in which all the relevant components, among which the transducer 21, and the positioning means 24a-24c are accommodated.

The use of the positioning means 24a-24c, in this case consisting of piezoelectric motors, makes it possible to realise a very precise orientation in the three-dimensional space of the ultrasound transducer 21 relative to the target region 1a. This principle is shown in FIG. 3. The ultrasound transducer 21 has a focal point F and is spaced from the target region 1a by a distance Y. According to the invention, the ultrasound transducer 21 is moved by the positioning means 24a-24c over a distance such that at least the ultrasound transducer 21 is oriented in the direction of the target region 1a. Ultrasound having a specific wavelength and frequency is generated by suitably driving the various transducer elements of the phased array ultrasound transducer, such that the ultrasound being emitted is focussed over the distance Y at the location of the target region 1a.

The various positioning means 24a-24c as well as the ultrasound transducer 21 are driven on the basis of the dose plan as generated by the device, which dose plan—as already described above—is based on image data of the target region 1a as obtained by the imaging means 10 on the one hand and the dose-planning unit 28 (see FIG. 1) on the other hand.

This flexible manner of driving makes it possible to subject the target region 1a to very specific treatments by means of the ultrasound transducer 21. As shown in FIG. 4a, the target region 1a is located in the human or animal body 1 in a treatment space 40 that is bounded by an upper surface 40a and a lower surface 40b. The ultrasound being generated and emitted can be focussed (F) in dependence on the prescribed treatment scheme by suitably driving the various transducer elements of the phased array ultrasound transducer 21 and the positioning means 24a-24c.

Two possibilities for treatment are shown in FIGS. 4b and 4c, which are representations of a two-dimensional and a three-dimensional treatment, respectively. FIG. 4b shows a treatment method wherein the ultrasound transducer 21 is driven in such a manner that the focus F is displaced in layers through the target region 1a. Ultrasound from the ultrasound transducer 21 is thus applied to the target region 1a in slices (layers 30a-30f). The two-dimensional treatment method, too, can be carried out by using two types of ultrasound application. According to the first type, ultrasound is directed spot-wise at the specific slice of the target region la in each layer, with the single focus F being oriented at one or more positions in the same plane 30a-30b by suitably driving the various transducer elements of the phased array transducer 21 on the one hand and the various positioning means 24a-24c on the other hand. In the case of another ultrasound application type in the two-dimensional plane, a movement is imposed on the ultrasound transducer 21a such that the focal point F is spirally positioned in each plane 30a-30f.

According to another treatment method as shown in FIG. 4c, the focal point F of the ultrasound transducer is positioned in a three-dimensional space 31. This treatment method concerns the volume-wise application of ultrasound to the target region 1a by the ultrasound transducer 21, wherein the focus F is positioned volume-wise in the three-dimensional space 31 by driving the various transducer elements and the positioning means 24a-24c.

One application of the device according to the invention concerns the treatment of proliferative tissue in a female breast, for example, wherein the device can be mounted in a positioning or fixing frame 50 that is arranged for positioning or fixing at least one breast of a female patient, which functions as the target region 1a.

As is shown in FIG. 5, the positioning or fixing frame is made up of an upper surface 50a and a lower surface 50b, which, through the use of suitable spacers 50c, form a spatial construction comprising an interior space 52. The upper surface 50a functions as a supporting surface for the upper body of a female patient who, once present on the upper surface 50a, can position both breasts in the openings 50a and 50b in such a manner that the breasts extend into the interior space 52.

As a result of the compact construction of the device 20 (see FIG. 1 as well as FIG. 6a), the device as a whole can be moved between the two upper and lower plates 50a-50b in the interior space 52 from aside as a separate unit, in such a manner that the ultrasound transducer 21 will apply ultrasound in lateral direction to the target region 1a (the breast of the female patient) through the application window 20a. The lateral exposure of the target region la by the device according to the invention enables a more precise focussing of the ultrasound in the target region 1a and makes it possible to realise a more precise treatment scheme for the treatment of proliferative tissue in the target region 1a. The treatment schemes thus realised can be carried out more precisely and more efficiently, so that the chance of recovery increases considerably.

FIGS. 7a and 7b show a further embodiment of a device according to the invention.

More in particular, the embodiment as shown in FIGS. 7a and 7b discloses a device in which the positioning means 24a′-24b′ are capable of orienting the ultrasound transducer 21 relative to the target region in which the application of ultrasound is to take place.

As is shown in FIGS. 7a, the ultrasound transducer 21 is movably mounted in a frame 70 forming part of the device by means of a gyroscope-like construction, in such a manner that the ultrasound transducer 21 can be rotated about its two orthogonal axes 71a-71b by suitable rotary positioning means 24a′-24b′. In this way the ultrasound transducer can be oriented very precisely relative to the target region for the purpose of applying ultrasound for treating proliferative tissue. More specifically, the focal distance F (see FIG. 7b) can be oriented precisely relative to the target region in this manner.

1. A device for directing ultrasound at a target region in a human or animal body, in particular for treating proliferative tissue, comprising:
imaging means for generating one or more images of the target region in the body that is to be treated;
processing means for processing the obtained images;
at least one ultrasound transducer arranged for placement in direct proximity of the target region to be treated; and
driving means for driving said at least one ultrasound transducer on the basis of signals generated by the processing means, characterized in that the device is provided with positioning means for orienting the ultrasound transducer relative to the target region.

2. A device according to claim 1, characterized in that the positioning means comprise at least one excitable piezoelectric motor.

3. A device according to claim 2, characterized in that means for feeding back the current position of the motor to the driving means are provided near each piezoelectric motor.

4. A device according to claim 3, characterized in that said feedback means in particular comprise at least one strain gauge.

5. A device according to claim 1, characterized in that the device is provided with Peltier-type cooling means.

6. A device according to claim 1, characterized in that the device comprises a housing, in which at least the ultrasound transducer and the driving means are accommodated.

7. A device according to claim 6, characterized in that the housing is mounted in a positioning or fixing frame for positioning or fixing at least one breast of a female patient, which functions as the target region.

8. A device according to claim 7, characterized in that the ultrasound transducer can be mounted in the housing in such a manner that the transducer will direct ultrasound in lateral direction at the target region during operation.

9. A device according to claim 1, characterized in that the ultrasound transducer is a phased array transducer.

10. A device according to claim 1, characterized in that the ultrasound transducer is a multichannel transducer, in particular a 256-channel transducer.

11. A device according to claim 1, characterized in that the device comprises a dose-planning unit, wherein the positioning means can be driven by the driving means partially on the basis of the signals as generated and delivered to the driving means by the dose-planning unit.

12. A device according to claim 1, characterized in that the driving means are arranged for focusing the ultrasound transducer in predetermined planes relative to the target region.

13. A device according to claim 1, characterized in that the driving means are arranged for focusing the ultrasound transducer spirally relative to the target region.

14. A device according to (claim 1), charactized in that the driving means are arranged for volume-wise focusing of the ultrasound transducer relative to the target region.

15. A device according to claim 1, characterized in that the imaging means are magnetic resonance-type imaging means.
